(19) United States
(12) Patent Application Publication
(10) Pub. No.: US 2022/0160931 A1
(43) Pub. Date: May 26, 2022

(54) SELF-GELLING WOUND CONTACT MATERIALS WITH IODINE DELIVERY

(71) Applicant: T.J.Smith and Nephew,Limited, Hull (GB)

(72) Inventors: Anthony Colin Dagger, York (GB); Nicholas Charlton Fry, Pocklington, York (GB); Victoria Jody Hammond, Hull (GB)

(21) Appl. No.: 17/598,628

(22) PCT Filed: Apr. 1, 2020
(86) PCT No.: PCT/EP2020/059249

§ 371 Date: Sep. 27, 2021

(30) Foreign Application Priority Data
Apr. 12, 2019 (GB) 1905206.7

(51) Int. Cl. A61L 26/00; A61L 15/22

(52) U.S. Cl. CPC A61L 26/0052; A61L 26/0066; A61L 26/008; A61L 2400/06; A61L 2300/106; A61L 2300/404; A61L 15/225

(57) ABSTRACT

Disclosed embodiments relate to wound care materials and methods that comprise a self-gelling composition, configured to form a gel upon contacting fluid. The self-gelling composition is a substantially homogeneous mixture, and comprises fluid-absorbent particles loaded with iodine-based antimicrobial agent, a dehydrated hydrogel powder, and a polymer base, configured to allow dispensing the self-gelling composition from a syringe or tube and to prevent the self-gelling composition from gelling prior to contacting fluid. The gel performs one or more of the following tasks during a wear time, when applied at the wound: absorbing fluid or exudate, releasing iodine-based antimicrobial agent, achieving antimicrobial activities at the wound, remaining structurally integral and cohesive, protecting the wound from exposure to microorganisms, and facilitating healing. After a wear time of at least three days, the gel can be removed in two pieces or less and leave less than 5% residue by weight.

### BACKGROUND

### Technical Field

This application discloses materials and methods, such as self-gelling compositions, wound care gel dressings, their uses, and methods of wound treatment therewith. In some embodiments, the materials and methods comprise or utilize a self-gelling composition configured to form a gel dressing at the wound site for antimicrobial delivery. The self-gelling composition may be applied to the wound as a paste or ointment, alone or in combination with a wound dressing or other wound management option(s).

### Description of the Related Art

Molecular iodine is active against bacteria, fungi and viruses, rapidly penetrating microorganisms, damaging proteins, nucleotides and fatty acids, leading to cell death. Consequently, iodine has been incorporated into numerous patient products, for example Iodosorb Cadexomer Iodine gel by Smith & Nephew.

Increasingly there is a need for improved mechanisms of delivering an effective dose of iodine or other antimicrobials, in combination with use of a wound dressing, to a wound. Of particular interest are user-friendly wound dressing formats that maintain or enhance the activities of iodine or other antimicrobials.

Therefore, improved methods and techniques for delivering iodine or other antimicrobials to wounds are needed.

### SUMMARY

Embodiments of the present disclosure relate to materials, methods and systems for wound treatment. Some disclosed embodiments relate to materials, methods, and systems for delivering iodine or other antimicrobials to a wound. It will be understood by one of skill in the art that application of the materials, methods, and systems described herein are not limited to a particular tissue or a particular injury.

Some of the embodiments described herein provide a self-gelling composition. The self-gelling composition may comprise, by weight, about 10% to about 50% fluid-absorbent particles, about 20% or more dehydrated hydrogel powder, and about 40% to about 70% polymer base. The fluid-absorbent particles may comprise crosslinked polysaccharide and an iodine-based antimicrobial agent encapsulated therein. The dehydrated hydrogel powder may comprise hydroxy derivatives of alkyl polymethacrylates. The dehydrated hydrogel powder can be configured to assemble into a polymeric network upon contacting fluid and, preferably, incorporate the fluid-absorbent particles into the polymeric network. The polymer base may comprise one or more polymers selected from the group consisting of polyethylene glycols (PEGs) and polypropylene glycols (PPGs). The self-gelling composition may comprise a substantially homogeneous mixture. The self-gelling composition can be configured to form a gel, comprising the polymeric network, within one hour upon contacting fluid.

The dehydrated hydrogel powder may comprise about 20% to about 30%, preferably, about 20% to about 25% by weight of the self-gelling composition. The hydroxy derivatives of alkyl polymethacrylates may comprise poly(2-hydroxy ethyl methacrylate) (pHEMA), poly(2-hydroxy propyl methacrylate) (pHPMA), or a combination thereof. The dehydrated hydrogel powder may comprise flakes of dehydrated hydrogel nano- or micron-sized particles.

The fluid-absorbent particles may preferably comprise about 10% to about 20% by weight of the self-gelling composition. The fluid-absorbent particles may comprise a diameter of less than 1 mm, preferably between 100 and 800 μm.

The polymer base may preferably comprise about 55% to about 70% by weight of the self-gelling composition. In some embodiments, the polymer base may comprise a mixture of a polyethylene glycol (PEG) and a polypropylene glycol (PPG) at a weight ratio between 1:1 and 1:10. The PEG may comprise an average molecular weight greater than 1,500 g/mole. The PPG may comprise an average molecular weight less than 1,200 g/mole. For example, the polymer base may comprise a mixture of PEG-4000 and PPG-425 at a weight ratio of 1:4 or, alternatively, a mixture of PEG-3000 and PPG-425 at a weight ratio of 1:9. In some other embodiments, the polymer base may comprise a mixture of PEGs of different molecular weights or, alternatively, a mixture of PPGs of different molecular weights.

Some embodiments of the self-gelling composition may be in the form of a paste or ointment. Some embodiments of the self-gelling composition is sterilized and within a syringe or tube.

Some of the embodiments described herein provide a wound dressing. The wound dressing may comprise a gel formed from a self-gelling composition, as described above or described elsewhere herein the specification, upon contacting a wound.

In the wound dressing of the immediately preceding paragraph, the gel can be configured to perform one or more of the following tasks during a wear time of about three days or more: The gel may absorb at least about 2.5 grams, preferably, at least about 2.7 grams of fluid or exudate per gram of the corresponding self-gelling composition. The gel may release the iodine-based antimicrobial agent, for example, upon the fluid-absorbent particles coming into contact with fluid from the wound. The gel may achieve one or more of the following anti-microbial activities at the wound site: a speed of kill against microorganisms, a sustained kill against microorganisms, and a broad-spectrum kill against microorganisms. The gel may remain structurally integral and cohesive at the wound site.

In some embodiments, the gel can be configured to allow the wound dressing to be removed in two pieces or less, or preferably in one piece, after a wear time of about three days or more. Upon removal from the wound after a wear time of about three days, the wound dressing may leave less than 10%, preferably, less than 5%, or less than 2% of residue by weight of the self-gelling composition in the wound.

Some of the embodiments described herein provide a method of treating a wound. The method of treating a wound may comprise: dispensing a self-gelling composition as described above or described elsewhere herein the specification from a syringe or tube to the wound; and forming a gel covering the wound surface upon the self-gelling composition coming into contact with fluid. The method may further comprise releasing the iodine-based antimicrobial agent from the self-gelling composition, for example, upon the fluid-absorbent particles coming into contact with fluid from the wound; and removing the gel in two pieces or less after a wear time of about three days or more.

The method of treating a wound of the preceding paragraph may further comprise, after dispensing the self-gelling composition from a syringe or tube to the wound, separately positioning a secondary wound dressing over the self-gelling composition and adhering the secondary wound dressing to skin surrounding the wound. The secondary wound dressing may comprise an adhesive adhered to at least part of the lower surface. The secondary wound dressing may comprise a transmission layer and/or absorbent layer and a cover layer over the transmission layer and/or absorbent layer. The secondary wound dressing may have a perimeter shape that is substantially the same as or, alternatively, smaller than a perimeter shape of the cover layer.

Alternative or additional embodiments described herein provide a composition comprising one or more of the features of the foregoing description or of any description elsewhere herein.

Alternative or additional embodiments described herein provide a self-gelling composition comprising one or more of the features of the foregoing description or of any description elsewhere herein.

Alternative or additional embodiments described herein provide a wound dressing comprising one or more of the features of the foregoing description or of any description elsewhere herein.

Alternative or additional embodiments described herein provide a wound treatment system comprising one or more of the features of the foregoing description or of any description elsewhere herein.

Alternative or additional embodiments described herein provide a method of treating a wound comprising one or more of the features of the foregoing description or of any description elsewhere herein.

### DETAILED DESCRIPTION

### Overview

Embodiments described in this specification relate to formulations, materials and methods that comprise, incorporate, and/or utilize a self-gelling composition that undergoes a sol-gel transition and forms a gel upon contacting fluid or moisture. The self-gelling composition may be dispensed in the form of a paste or ointment to a wound from a syringe or tube. The gel formed from the self-gelling composition may be utilized as a stand-alone gel dressing. Alternatively, a secondary wound dressing may be separately positioned over the gel and adhered to skin surrounding the wound.

As used herein, the term “fluid” encompasses fluid exuding from the wound as well as any physiologically acceptable aqueous solution such as saline, sterile water, Ringer's solution, etc.

### Self-Gelling Composition

Some of the embodiments described herein provide a self-gelling composition. The self-gelling composition may comprise fluid-absorbent particles and dehydrated hydrogel powder. The self-gelling composition may further comprise a polymer base as a carrier material for dispersing the mixture of fluid-absorbent particles and dehydrated hydrogel powder. The self-gelling composition may be a substantially homogeneous mixture. The self-gelling composition may preferably be in the form of a paste or ointment. The self-gelling composition may be sterilized and within a syringe or tube.

The fluid-absorbent particles may comprise a crosslinked polymer. The crosslinked polymer may comprise a crosslinked polysaccharide. The fluid-absorbent particles may further comprise a therapeutic agent. The therapeutic agent may comprise an iodine-based antimicrobial agent. One of skill in the art will understand that at least a portion of the therapeutic agent, encapsulated within the fluid-absorbent particles, may comprise extractable therapeutic agent, and that the extractable therapeutic agent can be released from materials made from the self-gelling composition. One of skill in the art will also understand that the therapeutic agent, such as any disclosed herein this “Self-Gelling Composition” section or elsewhere in the specification, may be loaded within the fluid-absorbent particles in powder form. The fluid-absorbent particles may comprise spherical beads. The fluid-absorbent particles may comprise a diameter of less than 1 mm, preferably between 100 and 800 μm. The fluid-absorbent particles may be selected from a group comprising Cadexomer Iodine, Sephadex, Dextranomer, Debrisan, or a mixture thereof. The fluid-absorbent particles can be configured to swell upon contact with fluid or moisture and/or to release the extractable therapeutic agent.

In certain embodiments, the dehydrated hydrogel powder may comprise hydroxy derivatives of alkyl polymethacrylates. The hydroxy derivatives of alkyl polymethacrylates may comprise poly(2-hydroxy ethyl methacrylate) (pHEMA), poly(2-hydroxy propyl methacrylate) (pHPMA), or a combination thereof. The hydroxy derivatives of alkyl polymethacrylates may be crosslinked. The dehydrated hydrogel powder may comprise flakes of dehydrated hydrogel nano- or micron-sized particles. The nano- or micron-sized particles may be spherical, non-spherical, or a mixture thereof. The dehydrated hydrogel powder of the self-gelling composition can be configured to assemble into a polymeric network upon contacting fluid or moisture and, preferably, to incorporate the fluid-absorbent particles into the polymeric network.

In some embodiments, the polymer base may comprise one or more polymers selected from the group comprising polyethylene glycols (PEGs) and polypropylene glycols (PPGs). In some embodiments of the self-gelling composition, the polymer base may comprise a mixture of a polyethylene glycol (PEG) and a polypropylene glycol (PPG). The PEG may comprise an average molecular weight greater than 1,500 g/mole, and the PPG may comprise an average molecular weight less than 1,200 g/mole. The higher molecular weight PEG and the lower molecular weight PPG may be mixed at a weight ratio between 1:1 and 1:10. For example, in certain preferable embodiments, the polymer base may comprise a mixture of PEG-4000 and PPG-425 at a weight ratio of 1:4. As another example, in certain other preferable embodiments, the polymer base may comprise a mixture of PEG-3000 and PPG-425 at a weight ratio of 1:9. One of skill in the art will understand that the average molecular weight (g/mole or Da) of a PEG (or PPG) may be denoted as a number in the name of the PEG (or PPG). For example, PEG-400 refers to a PEG having an average molecular weight of approximately 400 g/mole (or Da). The polymer base can be configured to achieve one or more of the following features in the self-gelling composition: The self-gelling composition may be water-miscible. The self-gelling composition may not swell or gel prior to contacting fluid or moisture. The self-gelling composition may be dispensable from a container; and the container may comprise a syringe, a tube, a bag, a pipette, or a vial.

In some embodiments, a self-gelling composition is disclosed that comprises fluid-absorbent particles, dehydrated hydrogel powder and a polymer base. The fluid-absorbent particles may comprise about 10% to about 90%, about 10% to about 80%, about 10% to about 70%, about 10% to about 60%, about 10% to about 50%, about 10% to about 40%, about 10% to about 30%, or preferably about 10% to about 20% by weight of the composition. The dehydrated hydrogel powder may comprise about 10% or more, about 20% or more, about 20% to about 60%, about 20% to about 50%, about 20% to about 40%, about 20% to about 30%, or preferably about 20% to about 25% by weight of the composition. The polymer base may comprise about 20% to about 90%, 20% to about 80%, about 30% to about 80%, about 30% to about 70%, about 35% to about 70%, about 40% to about 70%, about 45% to about 70%, about 50% to about 70%, or preferably about 55% to about 70% by weight of the composition.

In some embodiments of the self-gelling composition of the immediately foregoing paragraph, the fluid-absorbent particles may comprise crosslinked polysaccharide and an iodine-based antimicrobial agent encapsulated therein, preferably, Cadexomer Iodine. The fluid-absorbent particles may each comprise between 0.1% and 5%, or between 1% and 2%, or preferably less than 2% by weight antimicrobial iodine. The total antimicrobial iodine, as encapsulated within the fluid-absorbent particles, may comprise about 50% by weight extractable iodine. In certain embodiments, the total antimicrobial iodine encapsulated within the fluid-absorbent particles may comprise about 10% to 90%, 20% to 80%, 30% to 70%, or about 40% to 60%.

Certain embodiments of the self-gelling composition, for example, may comprise, by weight, about 10% to about 15% fluid-absorbent particles, about 20% dehydrated hydrogel powder, and about 65% to about 70% mixture of PEG-4000 and PPG-425 at a weight ratio of about 1:4.

As another example, certain other embodiments of the self-gelling composition may comprise, by weight, about 15% fluid-absorbent particles, about 25% dehydrated hydrogel powder, and about 60% mixture of PEG-3000 and PPG 425 at a weight ratio of about 1:9.

One of skill in the art will understand that the self-gelling composition disclosed herein this “Self-Gelling Composition” section or elsewhere in the specification can be sterilized, for example, using gamma irradiation, without substantially changing the chemical and physical properties. Other embodiments of the self-gelling composition may be prepared by using sterile manufacturing methods.

### Gel Dressing

Some of the embodiments described herein provide a wound dressing or gel dressing. The wound dressing or gel dressing may comprise a gel formed from a self-gelling composition, such as any disclosed in the foregoing “Self-Gelling Composition” section or elsewhere in the specification, upon contacting a wound.

One of skill in the art will understand that the self-gelling composition, such as any disclosed in the foregoing “Self-Gelling Composition” section or elsewhere in the specification, may undergo a sol-gel transition. In some preferable embodiments, the sol-gel transition may be triggered solely by contacting with fluid or moisture and not by shearing force, for example, shearing force applied when the self-gelling composition passes through a syringe or a tube. Certain embodiments of the self-gelling composition can be configured to form a gel within one hour upon contacting fluid or moisture. In certain embodiments, the self-gelling composition may be configured to form a gel within about: 1 minute, 5 minutes, 10 minutes, 30 minutes, 1 hour, 1.5 hours, 2 hours, 5 hours, or more than 5 hours. The gel may be formed in situ at a wound site and cover at least a part of the wound surface. During the sol-gel transition, the self-gelling composition may absorb an amount of fluid or exudate from the wound and, accordingly, may swell in size. The gel may comprise a molecular network that comprises the polymeric network assembled from the dehydrated hydrogel powder. Certain preferable embodiments of the self-gelling composition may form a substantially homogeneous gel, and the substantially homogeneous gel may remain substantially cohesive and homogeneous throughout a wear time. The wear time refers to the interval of time during which the gel is applied at the wound. The wear time may be designed to comprise about one day, about two days to about three days, or about three days, or preferably about three days or more, for example, about three days to about seven days. In some undesired embodiments, the gel may fall apart within the wear time as a result of the swelling.

Some embodiments of the gel dressing, formed from the self-gelling composition, such as any disclosed in the forgoing “Self-Gelling Compositions” section or elsewhere in the specification, may perform one or more of the following tasks during the designed wear time: absorbing fluid or exudate from the wound, releasing therapeutic agent such as iodine-based antimicrobial agent, achieving anti-microbial activities, remaining structurally integral and cohesive, protecting the wound from injury and/or exposure to microorganisms, and facilitating wound healing.

Some embodiments of the gel dressing may absorb at least about 2.5 grams, at least about 2.6 grams, at least about 2.7 grams, at least about 2.8 grams, at least about 2.9 grams, or at least about 3.0 grams of fluid or exudate per gram of the corresponding self-gelling composition. For example, certain embodiments of the gel dressing, formed from the self-gelling compositions comprising about 10% to about 15% by weight Cadexomer Iodine, may absorb more than 3.1 grams of fluid or exudate per gram of the corresponding self-gelling compositions.

Some embodiments of the gel dressing may release the therapeutic agent, such as the iodine-based antimicrobial agent. In some embodiments, the therapeutic agent may be released upon the fluid-absorbent particles coming into contact with fluid from the wound. The gel dressing may have a release profile, for example, with a maximal release rate occurring within the first 24 hours. In some embodiments, a desired release profile may be achieved by increasing one or more of the following: the amount of the antimicrobial agent loaded in each fluid-absorbent particle, the density of the fluid-absorbent particles in unit volume of the gel, and the thickness of the gel. One of skill in the art will understand that the density of the fluid-absorbent particles in unit volume of the gel may be increased by varying the composition and/or amount of the polymer base, for example, by varying the selection and ratio of PEG and PPG of the polymer base. One of skill in the art will also understand that increasing the amount (%) of the fluid-absorbent particles in the corresponding self-gelling composition may reduce the structural integrity or cohesiveness of the gel.

Some embodiments of the gel dressing may achieve one or more of the following anti-microbial activities: a speed of kill against microorganisms, a sustained kill against microorganisms, and a broad-spectrum kill against microorganisms. One of skill in the art will understand that the type and amount of antimicrobial loaded in the corresponding self-gelling composition may affect the antimicrobial activities.

Some embodiments of the gel dressing may remain structurally integral and cohesive at the wound site during the wear time of about three days, or about three days or more.

Some embodiments of the gel dressing may be removed in two pieces or less, preferably, in one piece from the wound after the wear time of about three days, or about three days or more. Upon removal from the wound, some embodiments of the gel dressing may leave less than 20%, less than 15%, less than 10%, or preferably less than 5%, or less than 2% of residue by weight of the corresponding self-gelling composition in the wound.

### Method of Treating a Wound

Some embodiments, disclosed herein the specification provide a method of treating a wound or locus. The method of treating a wound or locus may comprise dispensing a self-gelling composition, such as disclosed in the forgoing “Self-Gelling Composition” section or elsewhere in the specification, from a syringe or tube to the wound. The self-gelling composition may form a gel covering the wound surface upon the self-gelling composition coming into contact with fluid or moisture. The gel may release the therapeutic agent, such as the iodine-based antimicrobial agent. In some embodiments, the gel may release the therapeutic agent upon the fluid-absorbent particles coming into contact with fluid from the wound. The method of treating a wound or locus may further comprise removing the gel in two pieces or less, preferably, in one piece after the wear time of about three days, or about three days or more.

The method of the immediately above paragraph may further comprise one or more of the following steps: absorbing fluid or exudate from the wound, reducing the wound bioburden, achieving anti-microbial activities, protecting the wound from injury and/or exposure to microorganisms, and facilitating wound healing.

In some embodiments, the method further comprises wetting the self-gelling composition, or the gel formed from the self-gelling composition, with moisture or liquid. In some such embodiments, this wetting step may precede, or follow, or be performed substantially simultaneously with the step of dispensing the self-gelling composition to the wound. In some other such embodiments, the wetting step may precede or be performed substantially simultaneously with the step of removing the gel. In some embodiments, the moisture or liquid used in the wetting step may be suitable for application at the wound site. For example, the moisture or liquid used in the wetting step may be saline solution or saline spray.

The method of treating a wound or locus, as disclosed above in this “Method of Treating a Wound” section or elsewhere herein this specification may further comprise, after dispensing the self-gelling composition from a syringe or tube to the wound, separately positioning a secondary wound dressing over the self-gelling composition. The secondary wound dressing can be configured to form a seal to skin surrounding the wound, and the method of treating a wound or locus may yet further comprise adhering the secondary wound dressing to skin surrounding the wound. The secondary wound dressing may comprise a transmission layer and/or absorbent layer and a cover layer over the transmission layer and/or absorbent layer. The secondary wound dressing may have a perimeter shape that is substantially the same as or, alternatively, smaller than a perimeter shape of the cover layer.

### Terminology

Any patents and applications and other references noted above, including any that may be listed in accompanying filing papers, are incorporated herein by reference. Aspects of the disclosure can be modified, if necessary, to employ the systems, functions, and concepts of the various references described herein to provide yet further implementations.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the description of certain embodiments and may be defined by the claims. The language of the claims is to be interpreted broadly based on the language employed in the claims and not limited to the examples described in the present specification or during the prosecution of the application, which examples are to be construed as non-exclusive.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

Certain embodiments of the disclosure are encompassed in the claims presented at the end of this specification, or in other claims presented at a later date. Additional embodiments are encompassed in the following set of numbered embodiments:

Embodiment 1. A self-gelling composition, comprising:
about 10% to about 50% by weight fluid-absorbent particles, wherein the fluid-absorbent particles comprise crosslinked polysaccharide and an iodine-based antimicrobial agent encapsulated therein;
about 20% by weight or more dehydrated hydrogel powder, the dehydrated hydrogel powder comprising hydroxy derivatives of alkyl polymethacrylates, wherein the dehydrated hydrogel powder is configured to assemble into a polymeric network upon contacting fluid and to incorporate the fluid-absorbent particles into the polymeric network; and
about 40% to about 70% by weight polymer base, wherein the polymer base comprises one or more polymers selected from the group consisting of polyethylene glycols (PEGs) and polypropylene glycols (PPGs);
wherein the self-gelling composition is a substantially homogeneous mixture, configured to form a gel, comprising the polymeric network, within one hour upon contacting fluid.

Embodiment 2. The self-gelling composition of Embodiment 1, wherein the hydroxy derivatives of alkyl polymethacrylates comprise poly(2-hydroxy ethyl methacrylate) (pHEMA), poly(2-hydroxy propyl methacrylate) (pHPMA), or a combination thereof.

Embodiment 3. The self-gelling composition of Embodiment 1 or 2, wherein the dehydrated hydrogel powder comprises flakes of dehydrated hydrogel nano- or micron-sized particles.

Embodiment 4. The self-gelling composition of any one of the preceding embodiments, wherein the dehydrated hydrogel powder comprises about 20% to about 30%, preferably, about 20% to about 25% by weight of the composition.

Embodiment 5. The self-gelling composition of any one of the preceding embodiments, wherein the fluid-absorbent particles comprise a diameter of less than 1 mm, preferably between 100 and 800 μm.

Embodiment 6. The self-gelling composition of any one of the preceding embodiments, wherein the fluid-absorbent particles preferably comprise about 10% to about 20% by weight of the composition.

Embodiment 7. The self-gelling composition of any one of the preceding embodiments, wherein the polymer base comprises a mixture of a polyethylene glycol (PEG) and a polypropylene glycol (PPG) at a weight ratio between 1:1 and 1:10.

Embodiment 8. The self-gelling composition of Embodiment 7, wherein the PEG comprises an average molecular weight greater than 1,500 g/mole, and the PPG comprises an average molecular weight less than 1,200 g/mole.

Embodiment 9. The self-gelling composition of Embodiment 8, wherein the polymer base comprises a mixture of PEG-4000 and PPG-425 at a weight ratio of 1:4.

Embodiment 10. The self-gelling composition of Embodiment 8, wherein the polymer base comprises a mixture of PEG-3000 and PPG-425 at a weight ratio of 1:9.

Embodiment 11. The self-gelling composition of any one of Embodiments 1-6, wherein the polymer base comprises a mixture of PEGs of different molecular weights, or a mixture of PPGs of different molecular weights.

Embodiment 12. The self-gelling composition of any one of the preceding embodiments, wherein the polymer base comprises preferably about 55% to about 70% by weight of the composition.

Embodiment 13. The self-gelling composition of any one of the preceding embodiments, wherein the self-gelling composition is in the form of a paste or ointment.

Embodiment 14. The self-gelling composition of any one of the preceding embodiments, wherein the self-gelling composition is sterilized and within a syringe or tube.

Embodiment 15. A wound dressing, comprising a gel formed from a self-gelling composition of any one of the preceding embodiments upon contacting a wound.

Embodiment 16. The wound dressing of Embodiment 15, wherein the gel is configured to perform one or more of the following tasks during a wear time of about three days or more:
the gel absorbs at least about 2.5 grams, preferably, at least about 2.7 grams of fluid or exudate per gram of the corresponding self-gelling composition;
the gel releases the iodine-based antimicrobial agent;
the gel achieves one or more of the following anti-microbial activities at the wound site: a speed of kill against microorganisms, a sustained kill against microorganisms, and a broad-spectrum kill against microorganisms; and
the gel remains structurally integral and cohesive at the wound site.

Embodiment 17. The wound dressing of Embodiment 15 or 16, wherein the gel is configured to allow the wound dressing to be removed in two pieces or less after the wear time of about three days or more.

Embodiment 18. The wound dressing of any one of Embodiments 15-17, wherein the gel is configured to allow the wound dressing to be removed in one piece after the wear time of about three days or more.

Embodiment 19. The wound dressing of any one of Embodiments 15-18, wherein the wound dressing, upon removal from the wound after the wear time of about three days, leaves less than 10%, preferably, less than 5%, or less than 2% of residue by weight of the self-gelling composition in the wound.

Embodiment 20. A method of treating a wound comprising:
dispensing a self-gelling composition of any one of Embodiments 1-14 from a syringe or tube to the wound; and
forming a gel covering the wound surface upon the self-gelling composition coming into contact with fluid.

Embodiment 21. The method of Embodiment 20, further comprising releasing the iodine-based antimicrobial agent from the self-gelling composition.

Embodiment 22. The method of Embodiment 20 or 21, further comprising removing the gel in two pieces or less after a wear time of about three days or more.

Embodiment 23. The method of any one of Embodiments 20-22, wherein after dispensing the self-gelling composition from a syringe or tube to the wound, separately positioning a secondary wound dressing over the self-gelling composition and adhering the secondary wound dressing to skin surrounding the wound.

1. A self-gelling composition, comprising:
about 10% to about 50% by weight fluid-absorbent particles, wherein the fluid-absorbent particles comprise crosslinked polysaccharide and an iodine-based antimicrobial agent encapsulated therein;
about 20% by weight or more dehydrated hydrogel powder, the dehydrated hydrogel powder comprising hydroxy derivatives of alkyl polymethacrylates, wherein the dehydrated hydrogel powder is configured to assemble into a polymeric network upon contacting fluid and to incorporate the fluid-absorbent particles into the polymeric network; and
about 40% to about 70% by weight polymer base, wherein the polymer base comprises one or more polymers selected from the group consisting of polyethylene glycols (PEGs) and polypropylene glycols (PPGs);
wherein the self-gelling composition is a substantially homogeneous mixture, configured to form a gel, comprising the polymeric network, within one hour upon contacting fluid.

2. The self-gelling composition of claim 1, wherein the hydroxy derivatives of alkyl polymethacrylates comprise poly(2-hydroxy ethyl methacrylate) (pHEMA), poly(2-hydroxy propyl methacrylate) (pHPMA), or a combination thereof.

3. (canceled)

4. The self-gelling composition of claim 1, wherein the dehydrated hydrogel powder comprises about 20% to about 30% by weight of the composition.

5. The self-gelling composition of claim 4, wherein the dehydrated hydrogel powder comprises about 20% to about 25% by weight of the composition.

6. (canceled)

7. The self-gelling composition of claim 1, wherein the fluid absorbent particles comprise a diameter of about 100 to 800 μm.

8. The self-gelling composition of claim 1, wherein the fluid-absorbent particles comprise about 10% to about 20% by weight of the composition.

9. The self-gelling composition of claim 1, wherein the polymer base comprises a mixture of a polyethylene glycol (PEG) and a polypropylene glycol (PPG) at a weight ratio between 1:1 and 1:10.

10. The self-gelling composition of claim 9, wherein the PEG comprises an average molecular weight greater than 1,500 g/mole, and the PPG comprises an average molecular weight less than 1,200 g/mole.

11. The self-gelling composition of claim 10, wherein the polymer base comprises a mixture of PEG 4000 and PPG-425 at a weight ratio of 1:4.

12. The self-gelling composition of claim 10, wherein the polymer base comprises a mixture of PEG-3000 and PPG-425 at a weight ratio of 1:9.

13. The self-gelling composition of claim 1, wherein the polymer base comprises preferably about 55% to about 70% by weight of the composition.

14. (canceled)

15. (canceled)

16. (canceled)

17. A wound dressing comprising the gel of claim 1, wherein the gel is configured to perform one or more of the following tasks during a wear time of about three days or more:
the gel absorbs at least about 2.5 grams, preferably, at least about 2.7 grams of fluid or exudate per gram of the corresponding self-gelling composition;
the gel releases the iodine-based antimicrobial agent;
the gel achieves one or more of the following anti-microbial activities at the wound site: a speed of kill against microorganisms, a sustained kill against microorganisms, and a broad-spectrum kill against microorganisms; and
the gel remains structurally integral and cohesive at the wound site.

18. (canceled)

19. The wound dressing of claim 17, wherein the gel is configured to allow the wound dressing to be removed in one piece after the wear time of about three days or more.

20. The wound dressing of claim 17, wherein the wound dressing, upon removal from the wound after the wear time of about three days, leaves less than 10%, preferably, less than 5%, or less than 2% of residue by weight of the self-gelling composition in the wound.

21. The wound dressing of claim 20, wherein the wound dressing leaves less than about 5% of residue by weight of the self-gelling composition in the wound.

22. The wound dressing of claim 21, wherein the wound dressing leaves less than about 2% of residue by weight of the self-gelling composition in the wound.

23. A method of treating a wound comprising:
dispensing the self-gelling composition of claim 1 from a syringe or tube to the wound; and
forming a gel covering the wound surface upon the self-gelling composition coming into contact with fluid

24. The method of claim 23, further comprising releasing at least some of the iodine-based antimicrobial agent from the self-gelling composition.

25. The method of claim 23, further comprising removing the gel in two pieces or less after a wear time of about three days or more.

26. The method of claim 23, wherein after dispensing the self-gelling composition from a syringe or tube to the wound, separately positioning a secondary wound dressing over the self-gelling composition and adhering the secondary wound dressing to skin surrounding the wound.
